(19) United States
(12) Patent Application Publication
(10) Pub. No.: US 2025/0295298 A1
(43) Pub. Date: Sep. 25, 2025

(54) DEVICES, SYSTEMS, AND METHODS FOR MEDICAL LIGHTING

(71) Applicant: Boston Scientific Scimed, Inc., Maple Grove, MN (US)

(72) Inventors: Mesum Ali ZAIDI, Grayson, GA (US); Zachary BALSIGER, Saint Paul, MN (US); Naomi Leah BENSON, Minneapolis, MN (US); Abby CLARK, Clarendon Hills, IL (US); Kenzie GERMANSON, Wayzata, MN (US); Kennedy Alyssa KRUSE, Village of Lakewood, IL (US); Chukwuemeka George OCHIEZE, Westborough, MA (US)

(73) Assignee: Boston Scientific Scimed, Inc., Maple Grove, MN (US)

(21) Appl. No.: 19/086,469

(22) Filed: Mar. 21, 2025

Related U.S. Application Data
(60) Provisional application No. 63/568,642, filed on Mar. 22, 2024.

(51) Int. Cl. A61B 1/06; A61B 1/00; A61B 1/267

(52) U.S. Cl. CPC A61B 1/0684; A61B 1/00126; A61B 1/00133; A61B 1/2676

(57) ABSTRACT

A medical system may include a cable or wire, and a plurality of lighting assemblies coupled to the cable or wire. The plurality of lighting assemblies each may include (i) a lighting element and (ii) one or more anchoring features. In a first configuration, a first lighting assembly of the plurality of lighting assemblies may be a first distance along the cable or wire from a second lighting assembly of the plurality of lighting assemblies. In a second configuration, the first lighting assembly may be a second distance from the second lighting assembly. The second distance may be greater than the first distance.

### CROSS-REFERENCE TO RELATED APPLICATIONS

This application claims the benefit of priority under 35 U.S.C. § 119 from U.S. Provisional Application No. 63/568,642, filed Mar. 22, 2024, which is incorporated by reference herein in its entirety.

### TECHNICAL FIELD

Various aspects of this disclosure relate generally to systems, devices, and methods for medical lighting. In particular, aspects of this disclosure relate to systems, devices, and methods for deploying lighting elements inside a subject during a medical procedure.

### BACKGROUND

During medical procedures, such as bronchoscopies, a medical professional may insert a medical device into a patient and may guide the medical device through tortuous anatomy to position the medical device at a target treatment site in the patient. For example, a medical professional may insert a bronchoscope into lung passages to diagnose and/or treat the lungs. Accessing anatomy of small sizes, e.g., bronchioles of a lung, may present challenges to an operator due to, for example, low light conditions at the target treatment site.

### SUMMARY

Examples of the present disclosure relate to, among other things, systems, devices, and methods for deploying lighting elements inside a subject during a medical procedure. Each of the aspects disclosed herein may include one or more aspects of the features described in connection with any of the other disclosed aspects.

For example, a medical system may include a cable or wire, and a plurality of lighting assemblies coupled to the cable or wire. The plurality of lighting assemblies each may include (i) a lighting element and (ii) one or more anchoring features. In a first configuration, a first lighting assembly of the plurality of lighting assemblies may be a first distance along the cable or wire from a second lighting assembly of the plurality of lighting assemblies. In a second configuration, the first lighting assembly may be a second distance from the second lighting assembly. The second distance may be greater than the first distance.

Any of the systems, devices, and methods disclosed herein may include any of the following features. The one or more anchoring features may be configured to affix the plurality of lighting assemblies to an anatomical surface. The wire may be configured to transition from an elongated configuration to a contracted configuration. Each of the plurality of lighting assemblies may be fixed with respect to the wire. The wire may be a first wire, and the medical system may further include a second wire. The second wire and the first wire may be twisted together to form a deployment line. In the first configuration, a length of the deployment line may have a first number of twists, and in the second configuration, the length of the deployment line may have a second number of twists. The second number may be less than the first number. The plurality of lighting assemblies may be movably coupled to the wire. The medical system may further include a driver configured to pull the second lighting assembly proximally with respect to the first lighting assembly to transition from the first configuration to the second configuration. The lighting element may include one or more light emitting diodes (LEDs). The medical system may include a retention feature that is distal of a distalmost of the plurality of lighting assemblies. The medical system may include a bronchoscope. The wire and the plurality of lighting assemblies may be configured to be inserted into a working lumen of the bronchoscope. A first subset of the plurality of lighting assemblies may be configured to be removed from the working lumen in a configuration in which a second subset of the plurality of lighting assemblies may be affixed to an anatomical surface. At least one of the plurality of lighting assemblies may include at least one of (i) a unique marking or indication or (ii) an orientation reference feature. The one or more anchoring features may include at least one of hooks, barbs, protrusions, or prongs. At least one of the plurality of lighting assemblies may include a lumen through which the wire extends.

This disclosure further includes a medical system including a cable or wire, a first lighting assembly and a second lighting assembly coupled to the cable or wire, the first lighting assembly and the second lighting assembly each including (i) a lighting element and (ii) one or more anchoring features; and a driver configured to move the second lighting assembly proximally with respect to the first lighting assembly.

Any of the systems, devices, and methods disclosed herein may include any of the following features. The driver may be configured to move the second lighting assembly proximally in a configuration in which the first lighting assembly is affixed to an anatomical surface via the one or more anchoring features of the first lighting assembly. At least one of the first lighting assembly or the second lighting assembly may include an orientation reference feature including: a liquid; and a gas bubble within the liquid.

This disclosure also includes a method. The method may include inserting a medical device into a subject. The medical device may include a handle and a shaft extending distally from the handle, the shaft having a working lumen. The method may further include positioning a distal portion of the shaft at a target site, deploying a first illumination device from a working lumen of the medical device, affixing the first illumination device to an internal surface of a subject, and with the first illumination device affixed to the internal surface, retracting a second illumination device proximally within the working lumen.

Any of the systems, devices, and methods disclosed herein may include any of the following features. The first illumination device may be coupled to the second illumination device by a wire or a cable.

### BRIEF DESCRIPTION OF DRAWINGS

The accompanying drawings, which are incorporated in and constitute a part of this application, illustrate exemplary aspects of this disclosure and together with the description, serve to explain the principles of the present disclosure.

FIG. 1 illustrates an exemplary medical device, in accordance with some aspects of the present disclosure.

FIG. 2 illustrates an exemplary lighting system, in accordance with some aspects of the present disclosure.

FIGS. 3A-3D illustrate an exemplary lighting system and method for deploying the lighting system, in accordance with some aspects of the disclosure.

FIG. 4 illustrates an exemplary anchoring feature of the lighting systems disclosed herein, in accordance with some aspects of the disclosure.

FIG. 5 illustrates an exemplary orientation reference feature, in accordance with some aspects of the disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Proximal and distal directions are labeled with arrows marked “P” and “D”, respectively, in some of the Figures.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although bronchoscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, lungs, the urinary tract, or gastrointestinal tract. It will be appreciated that, unless otherwise specified, endoscopes, duodenoscopes, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein.

Features of the medical systems herein may enhance illumination in small body lumens, such as bronchioles. According to some aspects of the present disclosure, the medical system may include lighting assemblies (e.g., lights) attached to a deployment line (e.g., a wire or cable). The deployment line may be fed through a primary lumen of a medical device (e.g., a working channel of the medical device). At least some of the illuminators may be deployed distally from the primary lumen at a target site in a subject. Deployment of the line may involve use of a driving device to deploy illuminators at the target site in the subject. The illuminators may enhance visibility for an operator (e.g., a medical professional) performing a medical procedure with the medical device.

Reference will now be made in detail to examples of the present disclosure described above and illustrated in the accompanying drawings. Wherever possible, the same reference numbers will be used throughout the drawings to refer to the same or like parts.

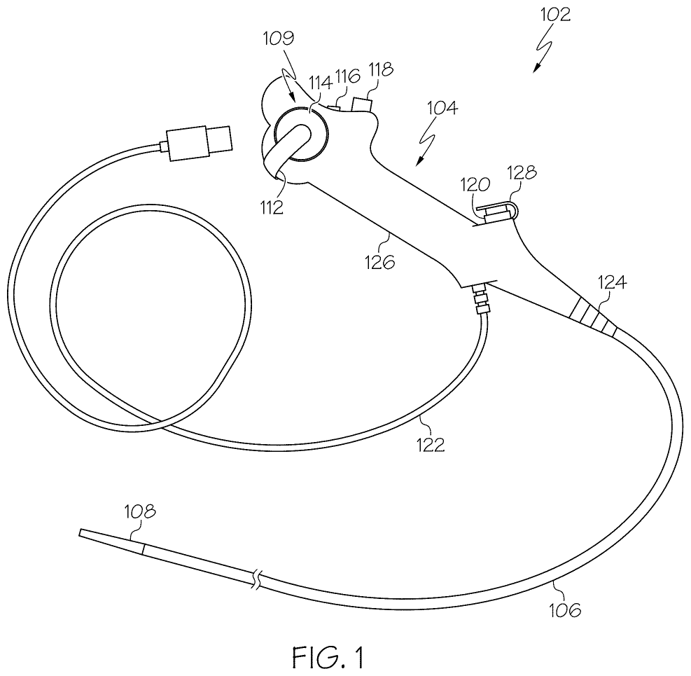

FIG. 1 illustrates an exemplary medical device 102 (e.g., a bronchoscope) having a handle 104 and a shaft 106 extending distally from handle 104. A stress relief portion 124 may bridge handle 104 and shaft 106. Shaft 106 may be at least partially flexible to facilitate navigation of shaft 106 through tortuous anatomical passages in the body. Shaft 106 may include a distal end portion that terminates at a distal tip 108. Distal tip 108 may include imaging devices (e.g., cameras, other components having imagers, and/or other optical elements such as lenses), and/or other electronic components (e.g., circuit board(s), capacitors, diodes, resistors, etc.), one or more light sources (e.g., LEDs, fiber optics, etc.), and an opening in communication with a working lumen (working channel) extending through shaft 106 and to a port 120 on handle 104. Electronic components of the devices described herein may include electrical connections, and may also include various elements mounted, for example, or otherwise connected to, the electrical connections.

In some aspects, distal tip 108 may feature a reflective coating such as silver to reflect light emitted by light sources (e.g., anchoring lights) towards a target site, rather than being absorbed by distal tip 108. Shaft 106 may define a longitudinal axis and may include a working lumen. An operator may remove a cap or seal 128 from port 120, and may insert a medical instrument or other device or system (such as the lighting systems described below) into port 120 and may extend the medical instrument or other device distally through the working lumen of medical device 102. Handle 104 may include a grip portion 126, which may allow an operator to grasp handle 104 during a medical procedure.

Handle 104 may include an actuation mechanism 109 having a lever 112 and a wheel 114 (e.g., a cam wheel) on handle 104 to facilitate articulation, steering, and/or deflection of shaft 106 and distal tip 108 (e.g., 180 degree articulation). Handle 104 may also include a button 116 for image capture (e.g., capture video and/or still images from the imaging device at distal tip 108) and a valve 118 to control suction, e.g., to provide air and/or water suction through the working lumen of medical device 102. Handle 104 may be coupled to an umbilicus 122. Umbilicus 122 may extend from handle 104 to one or more auxiliary devices. The one or more auxiliary devices may include a controller or control system, an imaging system, a power supply, a display, etc.

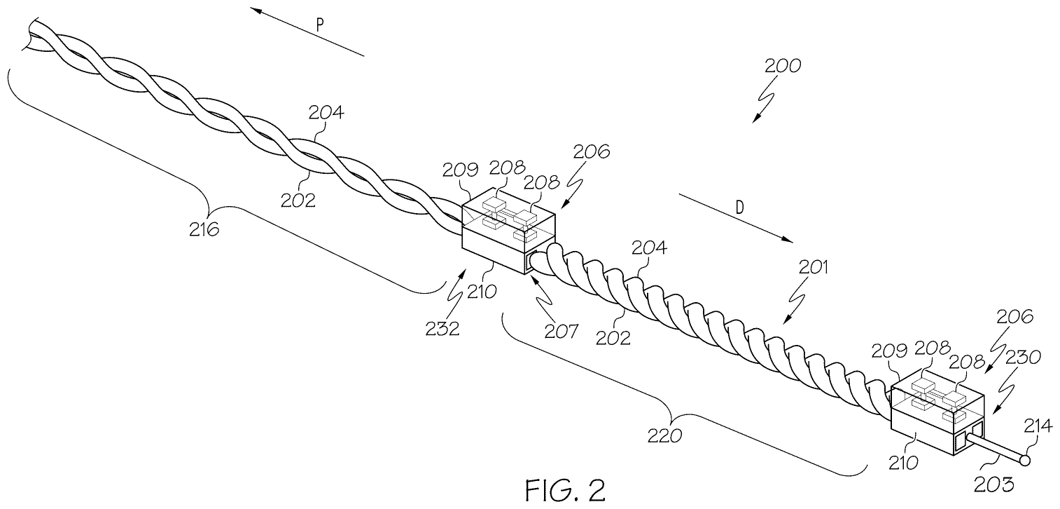

FIG. 2 illustrates an exemplary lighting system 200, in accordance with some aspects of the present disclosure. Lighting system 200 may include a deployment line 201 and a plurality of lighting assemblies 206. Lighting system 200 may be configured to be inserted into a working lumen of medical device 100. At least some of lighting assemblies 206 may be deployed from a distal opening of the working lumen, such that the lighting assemblies 206 are distal to distal tip 108.

Deployment line 201 may include a plurality of wires or cables, which may be or include conductive wires or cables. For example, as shown in FIG. 2, deployment line 201 may include a first cable or wire 202, a second cable or wire 204, and a third cable or wire 203. First wire 202, second wire 204, and third wire 203 may be twisted together, woven, or braided, or may extend approximately parallel to one another, in order to form deployment line 201. As shown in FIG. 2, first wire 202 and second wire 204 may be wound around third wire 203, such that third wire 203 may extend through a coil formed by first wire 202 and second wire 204. In some examples, both wire 202 and wire 204 may both be contained within a single sheath or harness.

As discussed above, third wire 203 may be positioned within or between first wire 202 and second wire 204. Wire 203 may be partially or fully enclosed by first wire 202 and second wire 204. For example, wire 203 may be surrounded by one or more twists, weaves, braids, helices, and the like formed by first wire 202 and second wire 204. Wire 203 may provide for shape memory properties of deployment line 201. In some aspects, wire 203 may be (or include) a nitinol wire, cable, or line that is approximately 0.05-0.1 mm in diameter (e.g., approximately 0.0762 mm in diameter), though this composition and diameter is only exemplary.

A protrusion 214 (e.g., a welded bead) may be at a distalmost end of third wire 203. Protrusion 214 may extend distally from a distalmost point of a distalmost of lighting assemblies 206 (e.g., from distal lighting assembly 230) and be affixed or otherwise attached to (e.g., integrally formed with) the end of wire 203. Protrusion 214 may prevent lighting assemblies 206 from slipping off third wire 203. Protrusion 214 may also be referred to as an arresting bead or a retention feature. Protrusion 214 may have a greater diameter than lumen 207 of lighting assemblies 206, to inhibit lighting assemblies 206 from moving distally past protrusion 214. Additionally or alternatively, protrusion 214 may be radiopaque or have other visualization properties to assist in positioning system 200.

First wire 202 and second wire 204 may have the same or differing properties. In an example, first wire 202 and second wire 204 may each be or include copper wire (and/or copper wire harnesses) that are approximately 44 gauge (approximately 0.0584 mm) in diameter. In some examples, first wire 202 and/or second wire 204 may provide energy to lighting assemblies 206. First wire 202 and second wire 204 may be helically twisted or coiled or otherwise bundled/bound together around third wire 203.

Together (and, optionally, separately), first wire 202, and second wire 204 (and, optionally, third wire 203 if it is also coiled or bent) are capable of longitudinal elongation and longitudinal contraction, such that at least a portion of deployment line 201 (e.g., a portion including first wire 202 and second wire 204) may lengthen and shorten. First wire 202 and second wire 204 may elongate and contract in tandem with one another (e.g., both first wire 202 and second wire 204 expand and contract in approximately the same manner). First wire 202 and second wire 204 may each maintain a state of elongation and/or contraction based on the elongation and/or compression of wire 203, which as mentioned above, may have shape memory properties. Thus, deployment line 201 may transition between an elongated configuration and a contracted configuration (e.g., from an elongated configuration to the contracted configuration). In the elongated configuration of deployment line 201, twists/coils of first wire 202 and second wire 204 around wire 203 may be lengthened and stretched out, such that adjacent twists/coils are separated from one another. In a contracted configuration of deployment line 201, twists/coils of first wire 202 and second wire 204 may be compressed toward one another, such that adjacent twists/coils are contacting one another or closer to one another than in the elongated configuration. In the elongated configuration, deployment line 201 may have fewer twists or coils per length unit (e.g., inch, centimeter, etc.) than in the contracted configuration. In other words, in the contracted configuration, deployment line 201 may have more twists or coils per length unit (i.e., along a given length of the deployment line 201). Thus, a number of twists of deployment line along a given length of deployment line 201 may be greater in the contracted configuration and less in the elongated configuration.

Although three wires 202, 203, and 204 are referenced above, it will be appreciated that deployment line 201 may include only two wires or cables or only one wire or cable. In such an example, deployment line 201 may include one of first wire 202 or second wire 204 in addition to wire 203 such that it may be elongated or contracted in a manner similar to that discussed above. Alternatively, deployment line 201 may include only third wire 203. Third wire 203 may be coiled or may be uncoiled.

Lighting system 200 may include one or more lighting assemblies (or illumination devices) 206. Each lighting assembly 206 may include one or more circuit boards 210. Any of the one or more circuit boards may be a printed circuit board (PCB). It should be understood by one of ordinary skill in the art that while a circuit board is referenced in FIG. 2, circuit board 210 may be any suitable electronic circuitry capable of electrically connecting and controlling one or more lighting elements 208, such as light emitting diodes (LEDs). Circuit board 210 may be electrically connected to at least one of the wires 202 or 204 (or another element of deployment line 201, such as a single wire or cable of deployment line 201). Circuit board 210 may be approximately 0.5-0.8 mm in length (e.g., approximately 0.65 mm in length) measured longitudinally along a longitudinal axis of lighting system 200, though this is only exemplary. As shown in FIG. 2, circuit board 210 and/or other elements of lighting assemblies 206 (e.g., a housing or base of lighting assemblies 206) may have a lumen 207 extending longitudinally through lighting assembly 206.

Circuit board 210 may include one or more lighting elements 208 (e.g., LEDs). Shown on each circuit board 210 are two lighting elements 208. It should be understood that the quantity of lighting elements 208 on each circuit board 210 of lighting system 200 may vary and the depiction of two lighting elements 208 is only exemplary. Lighting elements 208 may be electrically connected to one or more traces or wires of circuit board 210. In some aspects of the disclosure, a transparent or translucent encapsulation or cover 209 may enclose lighting elements 208. Dimensions of cover 209 may correspond with the dimensions of circuit board 210.

As shown in FIG. 2, lighting assemblies 206 may have a rectangular prism shape. However, such a shape is merely exemplary. In some aspects, lighting assemblies 206 may be substantially spherical, hemispherical, irregular shape, or any other suitable shape. In some aspects, each lighting assembly may feature a distinct geometric marker or indication that may be detected via a visualization device such as a camera or an ultrasound. For example, lighting assemblies 206 (e.g., circuit boards 210) may include a fiducial or other radiopaque marker. In some examples, each lighting assembly 206 may include a differently shaped (e.g., unique) marker for visualization on radiography or ultrasound.

At least one of first wire 202 and second wire 204 may extend at least partially through lumen 207. At least one of first wire 202 and second wire 204 may electrically connect to each of circuit boards 210, such that at least one of first wire 202 or second wire 204 is electrically coupled to lighting elements 208. For example, at least one of first wire 202 or second wire 204 may be electrically coupled to interior portions of circuit boards 210. Wire 203 may pass through interior portions of lighting assemblies 206 (not shown). Lighting assemblies 206 may be affixed to wire 203. Alternatively or additionally, lighting assemblies 206 may be slideably affixed to wire 203.

Lighting assemblies 206 may be positioned along deployment line 201. In some examples, lighting assemblies 206 may be separated from one another by a portion of deployment line 201. For example, as shown in FIG. 2, a distal lighting assembly 230 may be separated by a proximal lighting assembly 232 by a first portion 220 of deployment line 201. A second portion 216 of deployment line 201 may be proximal of the proximal lighting assembly 232. Additional lighting assemblies 206 (not shown in FIG. 2) may be proximal of the proximal lighting assembly 232. As shown in FIG. 2, second portion 216 may have an elongated configuration, described above. First portion 220 may have a contracted configuration, described above. In some examples, the contracted configuration may be the biased, natural configuration of deployment line 201.

In alternatives, lighting assemblies 206 may lack an active lighting element 208 and may instead include a reflective material. Lighting from distal tip 108 of medical device 100 may reflect from lighting assemblies 206, thereby increasing visibility within a body lumen. In further alternatives, lighting assemblies 206 may include both reflective material and active lighting element(s) 208.

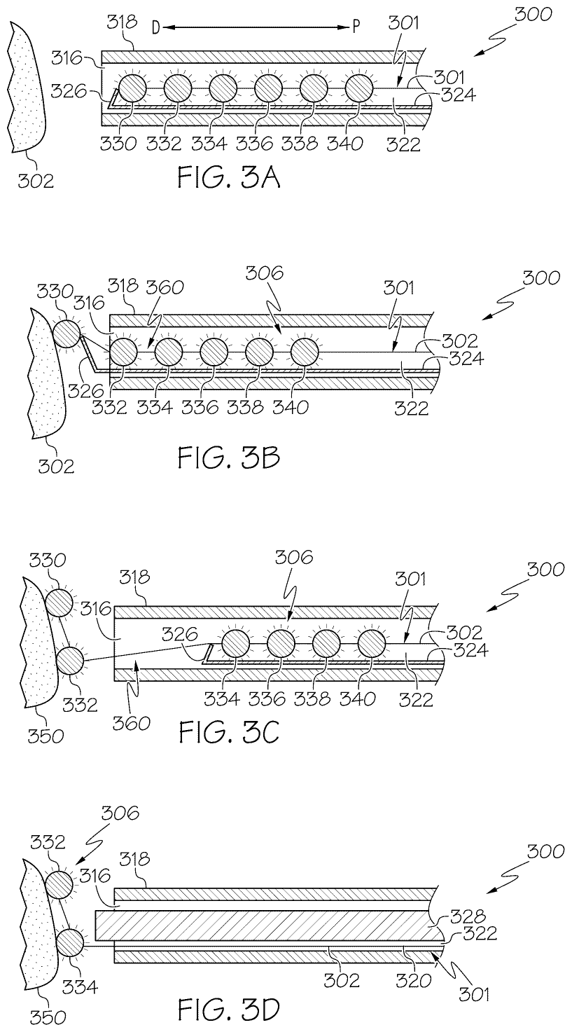

FIGS. 3A-3D illustrate another lighting system 300. Lighting system 300 may have any of the features of lighting system 200, unless otherwise specified. Furthermore, the methods and features described below may be used along with lighting system 200. FIGS. 3A-3D show a cross-sectional view of a working lumen 322 of medical device 100 and lighting system 300. Working lumen 322 may be defined by walls 318. Also shown in FIGS. 3A-3D is an anatomical surface 350, which may be an internal surface of the subject (a surface of an internal body lumen). Anatomical surface 350 may be located at a target site of interest to an operator. Anatomical surface 350 may be, for example, an interior surface of a bronchiole or another bodily lumen. An operator may guide distal tip 108 of medical device 100 to a target site through tortuous anatomy of a subject. As shown in FIGS. 3A-3D, lighting system 300 may be disposed within working lumen 322 and may be deployed distally from a distal opening 316 of working lumen 322.

Lighting system 300 may include a deployment line 301. Deployment line 301 may include any of the features of deployment line 201. Deployment line 301 may include a cable or wire 302. Although deployment line 301 is depicted as including a single wire 302, deployment line 301 may include a plurality of wires, such as third wire 203, first wire 202, and second wire 204. In some aspects, wire 320 may be a harness having one or more wires.

Coupled to wire 320 are a plurality of lighting assemblies 306, having any of the properties of lighting assemblies 206. FIG. 3A depicts six lighting assemblies 330, 332, 334, 336, 338, 340 (arranged distally to proximally); however, lighting system 300 may include any suitable number of lighting assemblies 306. It will be appreciated by one of ordinary skill in the art that the depiction of six lighting assemblies is only exemplary and that various numbers of lighting assemblies may be used with any of the exemplary aspects.

FIG. 3A shows lighting system 300 in a first configuration. For example, FIG. 3A may show lighting system 300 after it has been inserted into working lumen 322 once distal tip 108 is at a target site but before any of lighting assemblies 306 have been deployed to anatomical surface 350.

Further shown in FIG. 3A is a driver 324. Driver 324 may be inserted into working lumen 322 along with deployment line 301 and lighting assemblies 306 or separately from deployment line 301 and lighting assemblies 306. Driver 324 may be used by an operator to manipulate any of lighting assemblies 330, 332, 334, 336, 338, and 340. In some examples, the operator may use driver 324 to help to extend deployment line 301 and lighting assemblies 306 through working lumen 322 of shaft 106 and/or distal tip 108. A distal portion 326 of driver 324 may be hooked, barbed, notched, or otherwise shaped to engage a lighting assembly and manipulate the lighting assembly through and out of lumen 322. For example, distal portion 326 may be retroflexed in order to retain a distalmost lighting assembly 330 within lumen 322.

FIG. 3B shows lighting system 300 in a second configuration. In the second configuration, an operator may deploy one or more lighting assemblies 306 to anatomical surface 350.

In FIG. 3B, the operator may use distal portion 326 of driver 324 to manipulate the distalmost lighting assembly 306 (e.g., lighting assembly 330) and to attach lighting assembly 330 to anatomical surface 350. For example, distal portion 326 may be pivotable from the first configuration shown in FIG. 3A to the second configuration shown in FIG. 3B. In the first configuration of FIG. 3A, distal portion 326 may be angled proximally, whereas, in the second configuration of FIG. 3B, distal portion 326 may be angled distally.

Lighting assembly 330 may be attached to anatomical surface 350 by one or more anchoring mechanisms, which are described in greater detail below in FIG. 5. After placing/anchoring lighting assembly 306 to anatomical surface 350, the operator may place/anchor additional lighting assemblies, such as lighting assembly 332. It should be understood that it is possible to place and/or anchor lighting assemblies 306 in a non-distalmost-first manner. For example, an operator may place lighting assembly 332 and may then place lighting assembly 330. The operator may place any number of lighting assemblies, up to the number of total number of lighting assemblies coupled to deployment line 301. Deployment line 301 and/or driver 324 may have distance markings on a proximal portion (not shown) to enable an operator to understand where a next lighting assembly 306 to deploy is located, in addition to physical feedback to an operator via driver 324 (for example, via distal portion 326 making physical contact with a lighting assembly). Once a lighting assembly 306 is moved out of opening 316, an operator may use an imaging device (e.g., a camera) to view and manipulate the lighting assembly 306, deployment line 301, and/or driver 324.

FIG. 3C shows a third configuration of system 300. After deploying a desired number of lighting assemblies 306 (e.g., lighting assembly 330 and lighting assembly 332, as shown in FIG. 3B), an operator may retract, pull, or otherwise move the remaining lighting assemblies 334, 336, 338, and 340 with driver 324. Driver 324 may be transitioned to the first configuration of FIG. 3A. In some embodiments, an operator may apply a pulling force in a proximal direction to a distalmost unused lighting assembly (e.g., lighting assembly 334) using distal portion 326 of driver 324. As compared with a first configuration of FIG. 3B, a distance between lighting assembly 332 and lighting assembly 334 may be greater, measured along deployment line 301. Thus, lighting assembly 334 may be moved proximally relative to lighting assembly 332. As discussed below, this may be due to the fact that a length of a portion 360 of deployment line 301 is greater because (1) lighting assembly 334 has been moved proximally relative to deployment line 301, or (2) deployment line 301 has been elongated at portion 360.

Lighting assemblies 306 may be movably (e.g., slidably) coupled with respect to deployment line 301, like beads. For example, lighting assemblies 306 may include slidable electrical contacts that maintain electrical connections with deployment line 301 as lighting assemblies 306 move along deployment line 301. In this aspect, lighting assemblies 306 and/or circuit boards thereof may feature various locking mechanisms such as friction fits, magnetic locks, elastically deformable gaskets, and notches/protrusions to retain lighting assemblies 306 in a desired location until they are moved proximally.

Alternatively, lighting assemblies 306 may be frangibly fixedly coupled to deployment line 301, such that a sufficient proximal force on lighting assemblies 306 overcomes the frangible couplings, allowing lighting assemblies 306 to move proximally along deployment line 301.

Alternatively, as described with respect to deployment line 201, deployment line 301 may stretch longitudinally as proximal force is applied by driver 324. In such an example, each of lighting assemblies 306 may be fixedly coupled to deployment line 301 (fixed with respect to deployment line 301). As the operator applies a proximally-directed pulling force to lighting assembly 334 via driver 324, a portion of deployment line 301 proximal of lighting assembly 332 may elongate. In these aspects, lighting assemblies 306 may have a fixed position relative to deployment line 301. A distance between adjacent lighting assemblies may change 306 if a portion 360 of deployment line 301 between the adjacent lighting assemblies 306 is elongated, as discussed above.

It should be understood that anchoring features of lighting assemblies 330 and 332 may retain lighting assemblies 330 and 332 on anatomical surface 350 as an operator applies proximally directed force to lighting assemblies 334, 336, 338, and 340.

In FIG. 3D, lighting assemblies 330 and 332 have been deployed and are anchored to anatomical surface 350. Lighting assemblies 334, 336, 338, and 340 have been pulled proximally through lumen 322 by driver 324. Deployment line 301 may remain in lumen 322.

In some examples, a first subset of lighting assemblies 306 (lighting assemblies 334, 336, 338, and 340) may be withdrawn proximally through port 120 and removed from working lumen 322. A second subset of lighting assemblies 306 (lighting assemblies 330 and 332 may remain affixed to anatomical surface 350). An operator may insert an accessory medical instrument 328 such as, for example, biopsy forceps, graspers, baskets, snares, probes, scissors, retrieval devices, lasers, and other tools into lumen 322 and out of opening 316 to the target site and anatomical surface 350. Deployment line 301 may be sized and shaped so as to allow passage of medical instrument 328 through lumen 322. Lighting assemblies 330 and 332 may emit light in the target site without obstructing use of medical instrument 328 and/or manipulation of distal tip 108. Fiducials of lighting assemblies 306 may be visualized radiographically to assist with a procedure.

In some aspects, lighting system 300 may be configured to automatically increase and decrease brightness levels of light emitted by lighting assemblies 306. Alternatively, an operator may manually adjust aspects (e.g., brightness, color, etc.) of lighting assemblies 306.

At a conclusion of a medical procedure, an operator may remove lighting system 300 by, for example, pulling proximally on deployment line 301. Deployment line 301 may have sufficient strength that a proximal force on deployment line 301 may uncouple lighting assembl(ies) 306 from anatomical surface 350.

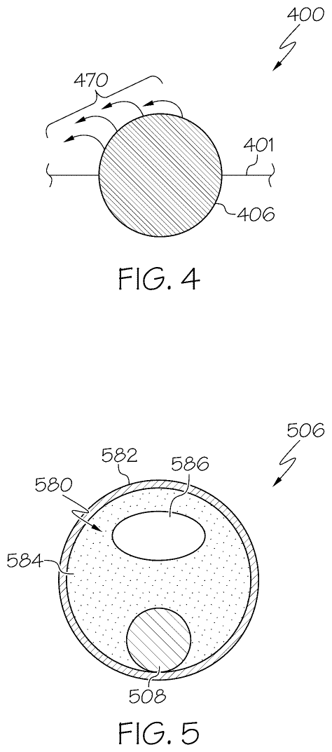

FIG. 4 shows a portion of an exemplary lighting system 400, having any of the properties of lighting systems 200, 300. Lighting system 400 includes a lighting assembly 406 which may have any of the properties of any of the previously described lighting assemblies, such as lighting assemblies 206, 306. Aspects of lighting assembly 406 may be incorporated into lighting assemblies 206, 306. Lighting assembly 406 is shown coupled to a deployment line 401, which may be substantially similar to any of the previously described deployment lines 201, 301. Lighting assembly 406 may feature one or more anchoring features 470. Anchoring features 470 may include hooks, barbs, protrusions, pegs, points, prongs, and the like to attach lighting assembly 406 to an anatomical surface, such as anatomical surface 350.

Anchoring features 470 may fix lighting assembly 406 relative to anatomical surface 350 and may be removable from anatomical surface 350 upon application of a sufficient force by an operator, as discussed above. An operator may manipulate lighting assembly 406 against an anatomical surface such that anchoring features 470 may engage and removably attach lighting assembly 406 to anatomical surface 350. Anchoring features 470 may be configured to keep lighting assembly 406 fixed with respect to anatomical surface 350 during retraction of other lighting assemblies 406 (as discussed above), while being removable upon application of a sufficient proximal force on lighting assembly 406 (e.g., via a proximal force on deployment line 401).

FIG. 5 illustrates a cross-sectional view of a lighting assembly 506, which may have any of the properties of lighting assemblies 206, 306, 406 (and vice versa). Lighting assembly 506 may include an orientation reference feature 580, in accordance with some aspects of the disclosure. Orientation reference feature 580 may be visualized (optically or by an ultrasound or radiographical device) to determine a reference direction (e.g., an “up” direction) by an operator.

For example, lighting assembly 506 may include a housing 582, which may include elements of orientation reference feature 580 and one or more lighting elements 508 (having any of the properties of lighting elements 208). Orientation reference feature 580 may include a liquid (e.g., saline) 584 inside housing 582. A gas bubble 586 may be within liquid 584. Gas bubble 586 may orient itself towards the “up” direction due to buoyancy within liquid 584.

In some examples, housing 582 may be at least partially transparent, and formed of a material such as a polymer or a glass. Thus, a camera of distal tip 108 may view gas bubble 586 to determine an orientation of lighting visually. Additionally or alternatively, liquid 584 and/or housing 582 may be able to be visualized on radiographs or by ultrasound. In some examples, a solid object that is denser than liquid 584 may be added to orientation reference feature 580 to similarly determine the “down” direction. In some examples, lighting element 508 may be visualized to identify a “down” direction. Any of the aforementioned reference features may be viewed with cameras and/or ultrasound to determine an orientation of lighting assemblies at the target site. In some aspects, orientation reference feature 580 may feature a magnetometer that may measure a magnetic field and/or magnetic dipole moment. The magnetometer may be used to determine orientation relative to a known source of magnetism.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

1. A medical system comprising:
a cable or wire; and
a plurality of lighting assemblies coupled to the cable or wire, the plurality of lighting assemblies each including (i) a lighting element and (ii) one or more anchoring features;
wherein, in a first configuration, a first lighting assembly of the plurality of lighting assemblies is a first distance along the cable or wire from a second lighting assembly of the plurality of lighting assemblies, and wherein, in a second configuration, the first lighting assembly is a second distance from the second lighting assembly, wherein the second distance is greater than the first distance.

2. The medical system of claim 1, wherein the one or more anchoring features are configured to affix the plurality of lighting assemblies to an anatomical surface.

3. The medical system of claim 1, wherein the wire is configured to transition from an elongated configuration to a contracted configuration.

4. The medical system of claim 3, wherein each of the plurality of lighting assemblies is fixed with respect to the wire.

5. The medical system of claim 3, wherein the wire is a first wire, and wherein the medical system further comprises a second wire, wherein the second wire and the first wire are twisted together to form a deployment line.

6. The medical system of claim 5, wherein, in the first configuration, a length of the deployment line has a first number of twists, and wherein, in the second configuration, the length of the deployment line has a second number of twists, wherein the second number is less than the first number.

7. The medical system of claim 1, wherein the plurality of lighting assemblies is movably coupled to the wire.

8. The medical system of claim 1, further comprising a driver configured to pull the second lighting assembly proximally with respect to the first lighting assembly to transition from the first configuration to the second configuration.

9. The medical system of claim 1, wherein the lighting element comprises one or more light emitting diodes (LEDs).

10. The medical system of claim 1, further comprising a retention feature that is distal of a distalmost of the plurality of lighting assemblies.

11. The medical system of claim 1, wherein the medical system includes a bronchoscope, and wherein the wire and the plurality of lighting assemblies are configured to be inserted into a working lumen of the bronchoscope.

12. The medical system of claim 11, wherein a first subset of the plurality of lighting assemblies is configured to be removed from the working lumen in a configuration in which a second subset of the plurality of lighting assemblies is affixed to an anatomical surface.

13. The medical system of claim 1, wherein at least one of the plurality of lighting assemblies includes at least one of (i) a unique marking or indication or (ii) an orientation reference feature.

14. The medical system of claim 1, wherein the one or more anchoring features comprise at least one of hooks, barbs, protrusions, or prongs.

15. The medical system of claim 1, wherein at least one of the plurality of lighting assemblies includes a lumen through which the wire extends.

16. A medical system comprising:
a cable or wire;
a first lighting assembly and a second lighting assembly coupled to the cable or wire, the first lighting assembly and the second lighting assembly each including (i) a lighting element and (ii) one or more anchoring features; and
a driver configured to move the second lighting assembly proximally with respect to the first lighting assembly.

17. The medical system of claim 16, wherein the driver is configured to move the second lighting assembly proximally in a configuration in which the first lighting assembly is affixed to an anatomical surface via the one or more anchoring features of the first lighting assembly.

18. The medical system of claim 16, wherein at least one of the first lighting assembly or the second lighting assembly includes an orientation reference feature including:
a liquid; and
a gas bubble within the liquid.

19. A method, comprising:
inserting a medical device into a subject, wherein the medical device includes a handle and a shaft extending distally from the handle, the shaft having a working lumen;
positioning a distal portion of the shaft at a target site;
deploying a first illumination device from a working lumen of the medical device;
affixing the first illumination device to an internal surface of a subject; and
with the first illumination device affixed to the internal surface, retracting a second illumination device proximally within the working lumen.

20. The method of claim 19, wherein the first illumination device is coupled to the second illumination device by a wire or a cable.
